(19) United States
(12) Patent Application Publication
(10) Pub. No.: US 2015/0335604 A1
(43) Pub. Date: Nov. 26, 2015

(54) Chromium and Chlorogenic Acid Weight Control Formulations

(71) Applicant: Boresha International, Pleasanton, CA (US)

(72) Inventors: Massood Moshrefi, Dublin, CA (US); Adib Nassar, Dublin, CA (US)

(21) Appl. No.: 14/720,564

(22) Filed: May 22, 2015

Related U.S. Application Data
(60) Provisional application No. 62/002,091, filed on May 22, 2014.
(60) Provisional application No. 62/097,884, filed on Dec. 30, 2014.

(51) Int. Cl. A61K 31/216; A23L 1/304; A23L 1/30; A23L 1/302; A23F 3/18; A61K 9/00; A61K 47/48; A61K 33/24; A61K 47/26; A61K 36/74; A61K 31/522; A61K 31/375; A23L 2/52; A23F 5/26

(52) U.S. Cl. CPC A61K 31/216; A23L 2/52; A23L 1/3045; A23L 1/3002; A23L 1/302; A23L 1/304; A23L 1/30; A23F 3/18; A23F 5/267; A61K 47/48076; A61K 33/24; A61K 47/26; A61K 36/74; A61K 31/522; A61K 31/375; A61K 9/0095; A23V 2002/00

(57) ABSTRACT

This invention provides weight loss formulations and methods of use. The formulations include a combination of chlorogenic acids and chromium compounds. The Cr/CGA combination can help reduce blood glucose, while fructose in the formulation provides quick energy and mitigates effects on appetite. The formulations can be introduced into any number of foods and beverages for consumption by dieters as part of their weight loss regimen.

### CROSS-REFERENCE TO RELATED APPLICATIONS

This application claims priority to and benefit of prior U.S. Provisional Application No. 62/002,091, by Massood Moshrefi, et al., filed May 22, 2014, and Provisional Application No. 62/097,884, by Massood Moshrefi, et al., filed Dec. 30, 2014. The full disclosure of the prior application is incorporated herein by reference.

### FIELD OF THE INVENTION

Formulations and methods of their use in weight control. The formulations include, e.g., a complementary combination of a chromium complex with chlorogenic acids and fructose. The combination reduces blood glucose and discourages conversion of sugars to fat, while inhibiting some appetite signals. The methods include use of the formulations in foods and beverages, such as coffee and tea, for consumption by dieters desiring weight control or weight loss.

### BACKGROUND OF THE INVENTION

It is desirable for people to maintain proper body weight as an aid to provide desired health, appearance, and fitness benefits. Weight loss can be aided by reduced appetite, reduced food consumption, burning more calories, and inhibition of lipid storage.

Some beverages have been presented as weight loss because they include no available calories. For example, there are a large number of beverages and foods on the market sweetened with no-calorie or low-calorie (e.g., artificial or indigestible) sweeteners. However, these foods and beverages typically fail to help a dieter control weight, e.g., because complex body feed-back systems override any benefit from the lower calorie serving. For example, it has been found that even though a sweet sugar-free drink may have no calories, the artificially sweetened beverage ultimately causes some persons to eventually take in more calories. One mode of action suggested for this phenomenon is that the sweet taste activates a pathway in the brain that signals the body to increase insulin levels and prepare the gut to receive a dose of sugars. When the sugars do not come, the excess insulin lowers blood glucose causing a food craving. When the artificially sweetened beverage drinker then eats more to ease the hunger, the body is ready to rapidly absorb calories from the gut, and the excess insulin is ready to rush sugars into adipose tissue for storage as fat.

In U.S. Pat. No. 5,480,657, to Allen, a composition of caffeine, fructose, and chromium is said to prevent or treat weight gain. However, unknown at the time, the formulation may have been less effective than hoped. For example, in some circumstances chromium delivery may have been significantly blocked, e.g., during beverage preparation. Further, the human body has a complex network of primary and alternate metabolic and anabolic pathways that may, e.g., reduce the effectiveness of an agent intended to aid in weight loss by modulating one pathway.

In view of the above, we have identified a need for weight loss formulations that are more recoverable through the steps involved in preparing and consuming the formulations in foods and beverages. We have seen that it would be desirable to have weight loss formulations that reduce the ability of the body to readjust back to a weight gaining mode in response to any particular weight loss agent. The present invention provides these and other features that will be apparent upon review of the following.

### SUMMARY OF THE INVENTION

Described herein are compositions, formulations, and methods for aiding people (and possibly other animals) in efforts to lose weight or in preventing weight gain. A combination of chromium and a chlorogenic acid can provide complementary benefits, beyond those of either alone, in reducing blood sugar and lipid storage. The weight-control benefits are further enhanced by inclusion of a low glycemic carbohydrate source, such as fructose, in the weight-control formulation. The formulations are useful compositions to incorporate into foods and beverages for dieters wishing to lose weight or avoid weight gain.

The weight loss formulations of the invention include at least chromium (Cr) and a chlorogenic acid (CGA). For example, a formulation active in facilitating weight loss can include a complex comprising chromium III (e.g., Cr⁺³) and comprising a niacin, a chlorogenic acid, and fructose. In preferred embodiments, the formulation complex further comprises a cysteine, e.g., a complex of chromium III with dinicocysteinate (CDNC). Optionally, chromium can be in the form of a complex with other types of amino acids besides cysteine, vitamins, as well as with picolinic acid, cinnamic acid, glucoronic acid and/or the like. Other ingredients that can contribute to the benefits of the formulation include, e.g., thermogenic agents, low glycemic carbohydrates, reducing agents, vitamins, amino acids, and trace metals.

In a typical serving of the formulation the chromium is present at a level from about 20 ug to about 200 ug (e.g., for a total in daily servings from about 50 ug to 500 ug) and CGAs present in the serving from about 50 mg to about 1000 mg (e.g., for a total in daily servings from about 100 mg to 2000 mg). There may be 1, 2, 3, 4, 5 or more servings per day. For example, a serving (e.g., dose) of the formulation can include from 100 to 300 ug of the complexed chromium, from 100 to 800 mg of the chlorogenic acid; and, from 100 mg to 8000 mg or more of fructose.

The chlorogenic acids (CGAs) can be provided from any suitable source, such as the CGAs present in green coffee bean extract. CGAs can be present in various forms, such as isomers, e.g., 3-O-caffeoylquinic acid (3-CQA), 4-O-caffeoylquinic acid (4-CQA), and 5-O-caffeoylquinic acid (5-CQA). In certain embodiments, additional benefits can be realized when the CGAs isomers are present in roughly equivalent levels (e.g., within +/−50% of each other). For example, 5-CQA can be provided in an amount less than the combined amount of 3-CQA and 4-CQA.

The formulation can include a low glycemic carbohydrate. For example, the formulation can include an uncooked carbohydrate, a high fiber carbohydrate source, a sugar alcohol, fructose, and/or the like. The fructose can comprise D-fructose and/or L-fructose. Fructose is preferred in many cases, e.g., for its sweetening character, ready availability as an energy source when blood glucose is reduced, digestibility, and the quite low glycemic index of 19.

The formulations can optionally include one or more thermogenic agents to increase consumption of calories. A preferred thermogenic agent is caffeine. In a typical food or beverage serving with the formulation there can be, e.g., from less than 20 mg to 200 mg of caffeine.

A method of preparing a weight loss beverage can include the steps of, e.g., 1) providing a composition of a complex comprising chromium III and comprising a niacin, a chlorogenic acid, and fructose; and, 2) blending the composition into a food or liquid beverage for human consumption. The blending can be, e.g., brewing with a coffee or a tea.

A person desiring to control body weight can consume the food or beverage.

As described herein, the formulation used in the methods of weight control can optionally include a chromium complexed with CDNC, caffeine, and/or chlorogenic acid in the form of a green coffee extract.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a CGA” can include a combination of two or more CGAs; reference to “a beverage” can include mixtures of beverages, and the like.

Unless defined otherwise, all technical and scientific terms used herein have the same meaning as commonly understood by one of ordinary skill in the art to which the invention pertains. Although any methods and materials similar or equivalent to those described herein can be practiced without undue experimentation based on the present disclosure, preferred materials and methods are described herein. In describing and claiming the present invention, the following terminology will be used in accordance with the definitions set out below.

As used herein, the term “complex” refers to a metal in the coordination center bound by complexing agents, such as coordinating molecules or ions. For example, Cr III coordinated by two surrounding niacins and a cysteine is a complex (Cr/CDNC).

As used herein “about” means in a range near the identified value, e.g., within 10%, 25%, or 50% of the identified value.

As used herein, the term “chlorogenic acid” refers to a member of the family of esters of hydroxycinnamic acids (caffeic acid, ferulic acid and p-coumaric acid) with quinic acid. For example, the ester of caffeic acid and quinic acid is a chlorogenic acid (e.g., one or more of 3-O-caffeoylquinic acid, 4-O-caffeoylquinic acid, and/or 5-O-caffeoylquinic acid).

As used herein, the term “green coffee extract” refers to an aqueous or polar extract of green (unroasted) coffee beans. One such green coffee extract is SVETOL™.

As used herein, the term “coffee” refers to coffee, as known in the art, and the common understanding. Coffee is the beverage brewed from the seeds (“bean”) of plants in the genus Coffea.

As used herein, the term “tea” refers to a beverage commonly prepared by water infusion of cured leaves of a tea plant in the genus of Camellia, particularly Camellia sinensis.

As used herein, the term “beverage” refers to a drink specifically prepared for human consumption. A weight loss beverage or weight control beverage is a beverage formulated to help reverse, avoid, or minimize accumulation of fat in the body of the drinker.

### DETAILED DESCRIPTION

The present inventions include formulations for use in foods and beverages useful in facilitating weight control. The formulations comprise a unique combination of components with a surprisingly complementary activities that work together to reduce appetite, consume energy, and reduce storage of sugars as fats.

We have found that compositions comprising, e.g., both a complexed soluble chromium III and a chlorogenic acid, e.g., in combination with the sugar fructose can provide surprisingly reduced blood glucose and insulin levels, while avoiding the increase in appetite usually associated with the reduced glucose levels. On ingestion by an animal (typically, a human being) the formulation can result in less food intake and reduced fat storage.

Additional formulation constituents can enhance the effectiveness of the weight loss formulation. For example thermogenic agents, anti-inflammation agents, and reducing agents can work in combination with the basic formulation to enhance efficacy, as will be discussed below.

### Weight Loss Formulations of Chromium and Chlorogenic Acid

The present formulations include combinations of ingredients that on ingestion reduce blood glucose. This results in a reduction of glucose transport into adipocytes, thus minimizing weight gain. Although lowered blood glucose would normally result in an increased appetite, this response can be neutralized by including in the formulation a significant amount low glycemic carbohydrate, such as fructose sugar. Consumption of energy and reduced appetite can optionally be further enhanced by inclusion of a thermogenic agent, such as caffeine in the formulation.

Chromium III is a component of the weight loss formulation with the activity of reducing blood glucose. Chromium (Cr) is a metal essential in at least trace amounts. Most chromium in the diet is never absorbed, but Cr which makes it through the intestinal walls can be picked up and transported in the blood stream by blood albumin. Transferrin can carry chromium into a cell, where it is complexed with a peptide called chromodulin. In this complex, chromium is thought to interact with the cell's insulin receptors making them more sensitive to insulin present in the blood. Activated insulin receptors in turn can activate certain transmembrane glucose transporter proteins which shuttle glucose into the cell, e.g., from the blood stream. Ultimately, the presence of chromium III is be associated with increased transport of glucose into cells and reduced levels of glucose in the blood stream, and reduced levels of insulin. Reduced insulin levels can reduce sugar storage as fat in adipose tissues.

Most chromium in the diet is never actually absorbed into the body. Although any form of absorbable chromium can functionally interact with other formulation constituents in the body, some forms work better than others. Chromium salts are poorly absorbed. Chromium picolinate (e.g., CHROMAX™) is somewhat better absorbed, but is not optimum in the context of the weight loss formulation and beverages. Chromium III complexed with three niacin molecules can also function in the formulation, but has some process and availability disadvantages with regard to solubility.

A preferred form of chromium in the weight control formulations is in a complex with two moles of niacin and one of cysteine. Such a complex is available as chromium III dinicocysteinate (CDNC), e.g., under the trade name of ZYCHROM™. We find that this form of chromium provides several advantages in the specific context of the present formulations. CDNC is generally recognized as safe, so is suitable for incorporation in an oral dosage formation. The chromium in CDNC is ultimately absorbed into the gut at a higher rate than other forms. The presence of cysteine provides an anti-inflammatory effect that may help counter the certain insulin resistance associated with oxidative stress.

Importantly, CDNC is relatively soluble in conditions of the gut and beverage processing. The enhanced solubility increases availability for transport from the gut into the body. In the context of product processing, CDNC has a higher percent recovery than other forms of chromium. For example, CDNC introduced into a process stream can be more likely to remain in solution or suspension regardless of parameters, such as pH, ionic strength, and the presence of adsorbing surfaces. Certain forms of chromium III may not be suitable for some uses of the weight loss formulations because inadequate amounts of the chromium may not ultimately make it into the food or beverage to be consumed by the end user. For example, the tri-niacin/Cr complex may not pass through filters during product processing. In the consumer product, the tri-niacin/Cr complex may be retained by the coffee grounds, tea, or filter paper and not be available in the prepared beverage ultimately consumed by the user. We find that availability of CDNC remains high through product manufacturing steps and beverage preparation steps in the hands of the consumer. This is typically not the case for the tri-niacin complex form of chromium III, which is not suitable for beverage application because it is not water soluble.

The weight control formulations can include chromium in an effective dose, when working in concert with other components of the formulation. For example, In the presence of from 100 to 800 mg of chlorogenic acid and 100 to 8000 mg of fructose, the formulation can include from less than 10 ug to more than 1000 ug of chromium, from 50 ug to 500 ug of chromium, from 100 ug to 300 ug of chromium, or about 200 ug of chromium. In a typical serving to a dieter, in combination with other formulation ingredients, chromium is provided in an amount of less than 20 ug to more than 2000 ug of chromium, from 50 ug to 1000 ug of chromium, from 100 ug to 500 ug of chromium, or about 200 ug of chromium. The dieter may partake, e.g., in from 1 to 10 servings per day, from 2 to 4 servings per day, or about 3 servings per day.

Chlorogenic acids (CGAs) are chemical compounds which are esters of caffeic acid and quinic acid. The term chlorogenic acids can also refer to a related family of esters of hydroxycinnamic acids (caffeic acid, ferulic acid and p-coumaric acid) with quinic acid. CGAs are important biosynthetic intermediates in plant biological systems, e.g., in lignin biosynthesis. CGAs are known have antioxidant effects.

(CGA) is a component of the weight loss formulation active in reducing blood glucose levels. CGA can influence glucose levels in a mode of action different from that of that of chromium, so can have surprisingly beneficial compounding effects. CGA can help reduce blood glucose levels by reducing absorption of sugars by the intestine, and by inhibiting glucose-6-phosphatase activity, e.g., in the liver.

Not being bound to any particular theory, we suspect the unexpected benefit of combining CGA and chromium may result from blocking alternate pathways. Carbohydrate metabolism is a complex and unpredictable web of enzyme pathways interacting with physiology. When one pathway is inhibited, alternate pathways may be utilized to an unforeseeable extent. The complementarity of chromium and CGA may result from one blocking an alternate route of another. For example, chromium alone may activate the insulin receptor, tending to remove blood glucose into adipocytes. However, the body's response may be to spur absorption of intestinal sugars, and/or release of sugar from the liver, among other balancing events. The addition of CGA can block part of the alternate pathways for increasing blood glucose so that the combination of chromium and CGA influences blood glucose levels more than either alone.

CGAs in the present formulations can come from any number of sources. A preferred source is green coffee bean extract, which is commercially available. Green coffee bean extract can be obtained by extracting unroasted (green) coffee beans with a polar solvent. Typically, it can be useful to first extract coffee oils from the beans, to enhance availability of the CGAs to the polar solvent. A commercially available green coffee extract, SVETOL™, can work well in the present formulations. Svetol includes three CGAs in a relatively similar amounts. For example, a preferred CGA composition can include 3-O-caffeoylquinic acid (3-CQA), 4-O-caffeoylquinic acid (4-CQA), and 5-O-caffeoylquinic acid (5-CQA) in roughly equivalent amounts (about the same amounts). Whereas many green coffee extracts are predominantly 5-CQA, preferred CGA components of the present formulations 5-CQA represents less than the combined amount of 3-CQA and 4-CQA; or each of the three isomers are present in amounts within 150% of each other.

The weight loss formulations can include CGAs in an effective dose, when working in concert with other components of the formulation. For example, in the presence of from 100 ug to 300 ug of chromium CGAs can be present in amounts ranging from less than about 30 mg to more than about 2000 mg, from 50 mg to 1000 mg, from 100 mg to 800 mg, from 200 mg to 600 mg, or about 300 mg of CGA. Further, the formulation can include 100 to 8000 mg of fructose and/or 20 mg to 200 mg of caffeine. In a typical serving, in combination with other formulation ingredients, CGAs are provided in an amount of 50 mg to more than about 2,000 mg, from 100 mg to 1,000 mg, from 150 mg to 600 mg, from 200 to 300 mg, or about 250 mg.

It is notable that although roasted coffee still contains some CGAs, the amount is typically reduced by roasting to below typical useful doses. Further, the character of the CGAs is modified by oxidative reactions and the proportions of isomers is changed. In an aspect of the invention, the present formulations are added to roasted coffee to provide an efficacious dose of CGAs, e.g., providing a cooperative combination with other formulation constituents, such as chromium III.

Fructose is an ingredient of the present formulations that works in concert with the powerful blood glucose reducing components. While chromium and CGAs reduce blood glucose in a dieter (person desiring to control or lose body weight), this raises the danger that the appetite of the dieter will increase. To counter this effect, the weight loss formulation includes a low glycemic component, such as fructose. Alternate low glycemic foodstuffs can optionally be used, such as, e.g., cooked or uncooked complex carbohydrates or high fiber foods.

Fructose is preferred, particularly in formulations intended for use in beverages. While glucose sets the standard with a glycemic index of 100, the glycemic index of fructose is only 19. Fructose is detected as sweeter than most other sugars, so less is required in a sweetened beverage. Fructose has the advantage that it does not stimulate insulin production or release. In the bloodstream, fructose can provide ready energy without providing a source of carbohydrate to readily feed into the production of fatty acids, e.g., in adipocytes.

In the present formulations, in the presence of from 100 ug to 300 ug of chromium CGAs and or 100 mg to 800 mg of CGAs, fructose can be present in amounts ranging from less than about 30 mg to more than about 12,000 mg, from 50 mg to 10,000 mg, from 100 mg to 8,000 mg, from 200 mg to 4,000 mg, from 500 to 1,000 mg, or about 800 mg. In a typical serving, in combination with other formulation ingredients, fructose is provided in an amount of 50 mg to more than about 10,000 mg, from 100 mg to 8,000 mg, from 500 mg to 6,000 mg, from 1000 to 3,000 mg, or about 2,000 mg.

Caffeine can be present in the present formulations, e.g., to provide a thermogenic and appetite suppressant effect. The stimulatory effect of caffeine can cause the body to burn additional calories over the course of a day. Alternately, the formulations can include no added caffeine.

In the present formulations, in the presence of from 100 ug to 300 ug of chromium, CGAs and/or 100 mg to 800 mg of CGAs, caffeine can be present in amounts ranging from less than about 10 mg to more than about 500 mg, from 20 mg to 200 mg, from 50 mg to 100 mg, or about 75 mg. In a typical serving, in combination with other formulation ingredients, caffeine is typically provided in an amount of less than 5 mg to more than about 800 mg, from 10 mg to 500 mg, from 20 mg to 200 mg, from 40 mg to 100 mg, or about 60 mg.

An alternate or additional thermogenic agent can be niacin. In many embodiments of the present formulations, niacin is present as an element of the chromium complex.

Optional cooperative ingredients to the weight control formulations can include reducing agents and trace metals. Cysteine can act as a reducing agent and anti-inflammatory complimenting the functions of the basic formulation ingredients of chromium and CGAs. Vitamin C can enhance the performance of the basic formulation, e.g., by mediating the blood glucose reductions effected by the chromium. Vitamin C can be present in the formulations in amounts per serving ranging from less than 5 mg to more than 1000 mg, from 10 mg to 500 mg, from 30 mg to 100 mg, or about 60 mg.

Beverages and food stuffs can benefit from receiving effective amounts of the present formulations. For example, the formulations can be present in servings of coffee, tea, juice drinks, diet drinks, dietetic foods, and/or the like. Servings of the weight loss formulations can be included in instant coffee, or in ground coffee for brewing into a weight loss coffee. Servings of the weight loss formulations can be included in instant tea, tea bags, or loose tea for brewing into a weight loss tea. The weight loss formulation can be incorporated into liquid diet compositions or into powders for dissolution or reconstitution.

### Methods of Using the Weight Loss Formulations

Methods of using the present formulations are an aspect of the present inventions. Weight loss formulations described herein can be prepared and incorporated into and food or drink intended for consumption. When the supplemented food or beverage is consumed, a person can experience reduced lipid storage and thermogenic effects, e.g., without concomitant increase in appetite.

Formulations can include, e.g., a chromium compound and a chlorogenic acid. Fat and carbohydrate metabolism includes broad and complex interactions of enzymes, signals and molecules. The Cr/CGA combination works surprisingly well in concert to block at least three avenues of glucose and fat metabolism to aid in weight loss.

The formulations can include fructose as an often preferred option. Fructose is readily transferred into the body from the gut and can provide energy and mitigate appetite signals that may result from the Cr/CGA blood glucose reduction.

The formulations can optionally include thermogenic agents (such as caffeine and niacin), reducing agents (such as vitamin C, vitamin E, and cysteine), and trace metals (such as Zn, Cu), and amino acids (such as arginine, GABA, and creatine).

The formulations can be prepared as a powder, or with properly proportioned ingredients in a liquid solution of suspension. The formulation can be blended into a food or beverage. Alternately, the formulation ingredients can be added separately to the food or beverage. Finally, the food and/or beverage can be consumed, e.g., in doses described herein, by a dieter desiring to reduce fat and lose weight.

### EXAMPLES

The following examples are offered to illustrate, but not to limit the claimed invention.

### Preparation of Weight Loss Formulations

Weight loss formulations are generally formulated with a target dosage of about 400 ug of chromium per day and 400 mg of a CGA per day. In addition Fructose is included to taste, but typically about 2 grams per serving (e.g., with 200 ug of chromium and 200 mg of a CGA). The formulation will optionally be formulated per serving with about 60 mg of caffeine.

In one example, a fruit drink was prepared with the following formula:

L-Arginine 5000 mg

Branch Chain Amino Acids (BCAA) 100 mg

Chlorogenic Acid 200 mg

Zychrome 0.72 mg

Folic Acid 0.50 mg

Vitamin C 60 mg

Acai Berry 50.00 mg

Zinc (Gluconate) 15.93 mg

Cu (Gluconate) 0.71 mg

Boron (calcium fructoborate) 37.04 mg

The formulation was provided as a dried powder. The powder was dissolved in 8 to 10 oz of water to provide a weight loss beverage. BCAA includes leucine, isoleucine and valine in a proportion of 2:1:1, respectively.

Coffee products can be provided including weight control formulations described herein. For example, a chromium complex (e.g., with niacin and cysteine) can be blended into the ground coffee beans. As CGAs are typically reduced below effective amounts by roasting, they can be added to the ground roast coffee beans. On coffee brewing with the ground roast, the Cr/CGA weight loss formulation will be present in the coffee for drinking. Optionally, fructose can be provided in the coffee grounds, or on the side to sweeten to taste. It is suggested the CGA be present in an amount of about 200 mg and chromium be provided as ZYCHROME™ (CDNC) in an amount of about 100 ug. Optionally, the Cr/CGA can be added to the coffee after brewing. The weight loss formulation can be provided in the same fashion with other drinks, such as, e.g., tea, cocoa, sport drinks, punches, soft drinks, and fruit drinks.

Another formulation for incorporation into food or beverages includes a complementary combination of chromium, CGA, caffeine, and vitamin C. For example, the following formulation has been prepared.

Chromium dinicocysteinate (9% Cr) 266.0 ug

Chlorogenic acid 200.0 mg

Caffeine 120.0 mg

Vit. C 120.0 mg

A formulation at or about these levels and proportions can be used to aid in weight control when consumed by a dieter.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A composition active in facilitating weight control, wherein the composition comprises:
a complex comprising chromium III and comprising one or more niacin;
one or more chlorogenic acid; and,
fructose.

2. The composition of claim 1, wherein the complex further comprises a cysteine.

3. The composition of claim 2, wherein the complex is chromium III complexed with dinicocysteinate (CDNC).

4. The composition of claim 1, wherein the chlorogenic acid comprises 3-O-caffeoylquinic acid (3-CQA), 4-O-caffeoylquinic acid (4-CQA), and 5-O-caffeoylquinic acid (5-CQA).

5. The composition of claim 4, wherein the amount of 5-CQA is less than the combined amount of 3-CQA and 4-CQA.

6. The composition of claim 4, wherein the chlorogenic acid is in the form of a green coffee bean extract.

7. The composition of claim 1, wherein the fructose comprises D-fructose or L-fructose.

8. The composition of claim 1, further comprising caffeine.

9. The composition of claim 1, further comprising instant coffee, tea, or ground coffee beans.

10. The composition of claim 1, further comprising vitamin C or trace metals.

11. The composition of claim 1, comprising the formulation of:
from 100 to 300 ug of the complexed chromium; from 100 to 800 mg of the chlorogenic acid; and, 100 to 8000 mg of fructose.

12. The formulation of claim 11, further comprising 20 to 200 mg of caffeine.

13. A method of preparing a weight loss beverage, the method comprising:
providing a composition comprising: a complex comprising chromium III and comprising a niacin; a chlorogenic acid; and, fructose;
blending the composition into a liquid beverage for human consumption.

14. The method of claim 13, wherein the complex comprises CDNC.

15. The method of claim 13, wherein the composition further comprises caffeine.

16. The method of claim 13, wherein the chlorogenic acid is in the form of a green coffee extract.

17. The method of claim 13, wherein said blending comprises brewing with a coffee or a tea.
